(19) United States
(12) Patent Application Publication
(10) Pub. No.: US 2014/0052026 A1
(43) Pub. Date: Feb. 20, 2014

(54) METHOD AND APPARATUS FOR MEDICAL DIAGNOSIS

(71) Applicant: AUGMENTED MEDICAL INTELLIGENCE LABS, INC., Cambridge, MA (US)

(72) Inventors: Andrew Mina Bishara, Cambridge, MA (US); Marc David Succi, Cambridge, MA (US); Fransiska Putri Wina Hadiwidjana, Surabaya (ID); Elishai Ezra, Jerusalem (IL)

(73) Assignee: AUGMENTED MEDICAL INTELLIGENCE LABS, INC., Cambridge, MA (US)

(21) Appl. No.: 13/970,423

(22) Filed: Aug. 19, 2013

Related U.S. Application Data
(60) Provisional application No. 61/684,629, filed on Aug. 17, 2012.

(51) Int. Cl. A61B 5/00

(52) U.S. Cl. CPC A61B 5/0053
USPC 600/587

(57) ABSTRACT

The present disclosure includes a medical device which can assist in detecting tissue stiffness. For example, the present disclosure includes a medical device which can detect tissue stiffness associated with cancerous tissue for detecting cancer in a patient. In addition, the medical device of the present disclosure can be used for conducting a clinical breast exam.

### REFERENCE TO PRIORITY DOCUMENT

This application claims the benefit of priority under 35 U.S.C. §119(e) of U.S. Provisional Patent Application Ser. No. 61/684,629, filed Aug. 17, 2012, under 37 C.F.R. §1.78(a). Priority of the filing date is hereby claimed and the full disclosure of the aforementioned application is incorporated herein by reference.

### FIELD

The subject matter described herein relates to embodiments of medical devices and methods for detecting properties of tissue, such as stiffness.

### BACKGROUND

Palpation can be used as part of a physical examination in which a part of a patient's body, such as an organ or area of tissue, is felt by the hands of a healthcare practitioner in order to determine one or more characteristics or properties related to that part of the patient's body. In some cases, palpation is used to detect painful areas and to qualify pain felt by the patient. Palpation can also be used for examining breast tissue, such as for detecting cancerous masses in the breast.

### SUMMARY

Disclosed herein are devices and methods related to embodiments of a medical device for monitoring physiological conditions. An embodiment of the medical device may include a glove body configured to fit over a hand of a user with at least one force sensor secured to at least one fingertip of the glove body. In addition, the medical device may include a displacement assessment device configured to collect data indicating displacement of at least one fingertip as it is pressed against tissue in order to determine the stiffness of the tissue. Additionally, the medical device may include a processor configured to process data obtained by the displacement assessment device to assess the stiffness of the tissue in contact with the at least one force sensor.

An embodiment of a method can include using a medical device for obtaining data characterizing stiffness of tissue and may include securing a medical device to a hand of a user. The medical device may include a glove body configured to fit over a hand of a user with at least one force sensor secured to at least one fingertip of the glove body, a displacement assessment device configured to collect data indicating displacement of at least one fingertip as it is pressed against tissue in order to determine the stiffness of the tissue, and a processor configured to process data obtained by the displacement assessment device to assess the stiffness of the tissue in contact with the at least one force sensor. In addition, the method may include placing at least one force sensor against the tissue of a patient and collecting data associated with the force sensor and data associated with displacement assessment device. Additionally, the method may include processing the data and determining the stiffness of the tissue.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

These and other aspects will now be described in detail with reference to the following drawings.

FIG. 1 shows an embodiment of a medical device configured to fit over a user's hand and detect stiffness of examined tissue.

FIG. 2 shows an example of a collection of data obtained by the medical device including data obtained from a force sensor and vibration sensor.

FIGS. 3A and 3B show an embodiment of the medical device showing force sensors positioned at distal ends of the middle finger, index finger and thumb of the glove body and a wrist enclosure.

FIG. 4 shows an embodiment of the medical device including nanotechnology sensors.

FIGS. 5 and 6 show an embodiment of the medical device including circumferential pressure sensors located along a part of the distal end, or fingertip, of the glove body for determining finger displacement relative to the examined tissue.

Like reference symbols in the various drawings indicate like elements.

### DETAILED DESCRIPTION

The present disclosure includes a medical device that can assist in detecting tissue stiffness. For example, the present disclosure includes a medical device that can detect tissue stiffness associated with cancerous tissue. In addition, the medical device of the present invention can use a technique similar to a clinical breast exam that can be used in places where some medical equipment and training, such as for conducting mammograms, are lacking, such as in developing countries.

Compared to the mammogram, the clinical breast exam can be much less resource intensive with comparable results. For example, clinical breast exams alone can find a substantial proportion of cancers while requiring fewer resources. However, clinical breast exam sensitivities and specificities can be varied, which can be at least partially attributed to differences in physician skill and patient physical characteristics.

The present medical device can provide cost-effective identification of tissue abnormalities, which may indicate cancerous tissue, such as cancerous nidus in a breast. While breast cancer is a prominent application, the medical device of the present disclosure can also apply to detecting any mass-based cancer near the skin surface, including testicular, prostate and thyroid cancer. In addition, other mass-based physical exam measurements, including pitting edema, sensing percussion based examinations, and determining the rough size of organs close to the surface of the skin (e.g. the liver) can be facilitated by this technology. The medical device of the present disclosure can also provide for quantification of clinical breast exam results.

In some embodiments, the medical device of the present disclosure can be composed of a fabric-based glove with at least one pressure or force sensor and associated circuitry secured to a part of the fingertips of the glove. The medical device can assist in detecting abnormalities underlying the skin via, as an example, a combination of pressure and force sensors on three fingers of the medical device.

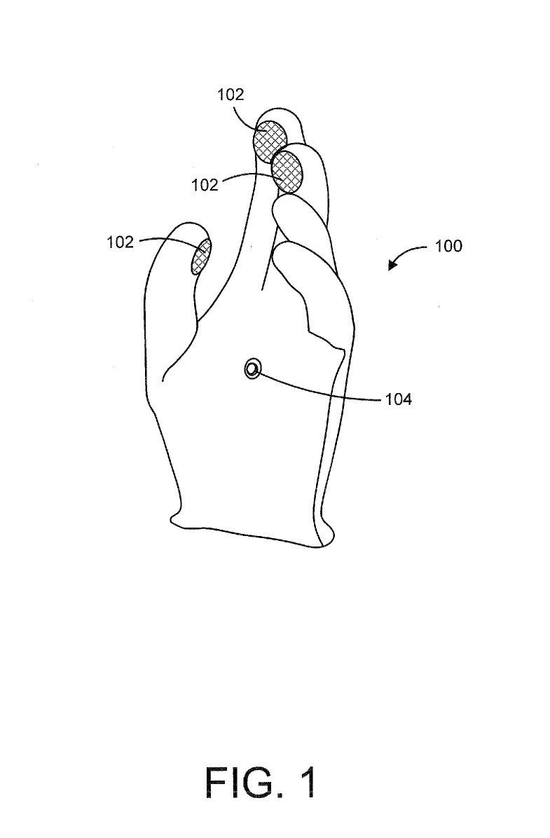

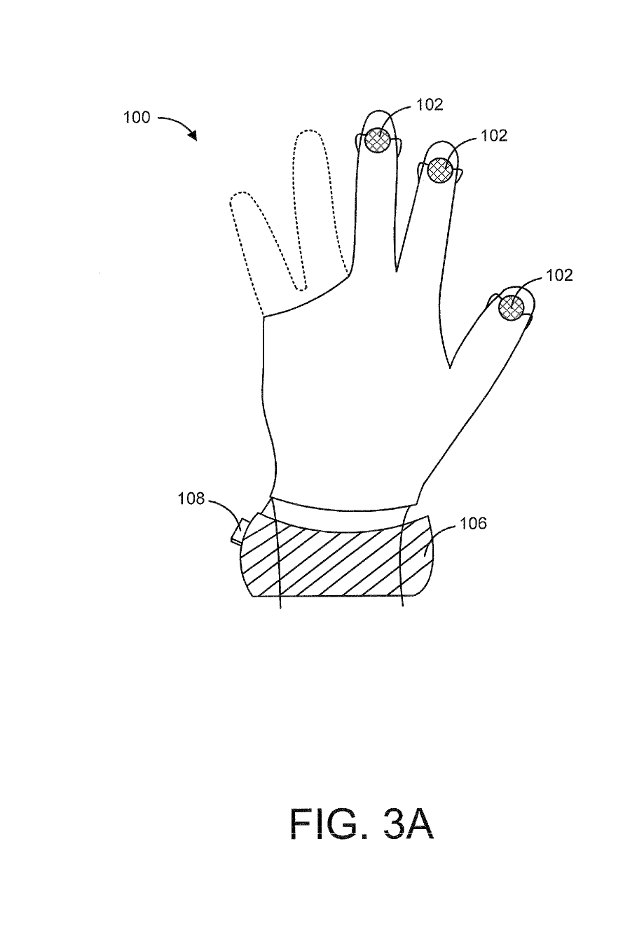

FIG. 1 shows an embodiment of a medical device 100 configured to fit over a hand and detect tissue stiffness, including detecting a variance in stiffness of examined tissue. The medical device 100 includes a glove body having force sensors 102 positioned at the distal end of the fingers, as shown in FIG. 1. In addition, the medical device 100 can include a vibration sensor or camera 104 positioned along the palm of the medical device 100. Additionally, the force sensors and vibration sensor may be coupled to a wrist enclosure 106, as shown in FIGS. 3A and 3B, which contains circuitry and electrical components for analyzing and transmitting data to a computer. The wrist enclosure 106 can include a USB connection 108, as shown in FIGS. 3A and 3B, for connecting a USB cable to the wrist enclosure 106 for transmitting data or may be configured for wireless data transfer.

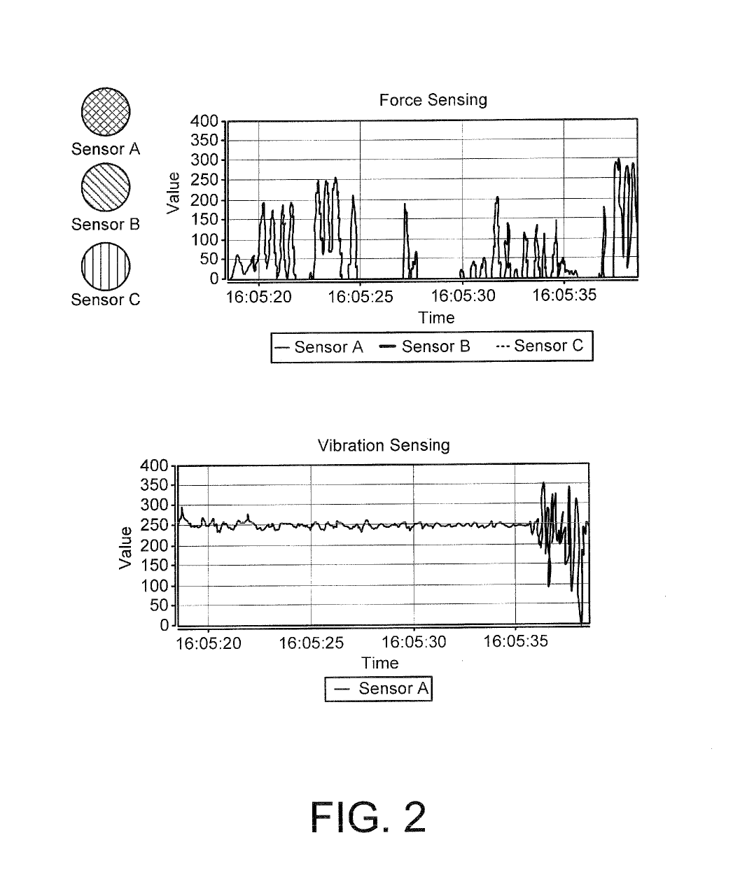

FIG. 2 shows an example of a collection of data obtained by the medical device of the present disclosure including data obtained from a force sensor and vibration sensor. These measurements can be taken from, for example, force sensors 102 positioned on distal ends of the index finger, middle finger and thumb of the glove body of the medical device 100, as shown in FIG. 1. Data obtained from these force sensors 102 can show relative values of force (y axis) and time (x-axis). As shown in FIG. 2, data can also be obtained from a vibration sensor and displayed in graphical form for analysis, such as by a physician. These measurements can assist a user in detecting the stiffness of the examined tissue in order to, for example, detect cancerous tissue.

Some embodiments of the medical device 100 can include sensors that can detect pressure, vibration, acceleration and temperature. In addition, the medical device can include sensors for electronic palpation, galvanic skin conductance sensors, various cameras, including a heat infrared camera, microphones and audible devices, such as buzzers or speakers.

FIGS. 3A and 3B illustrate an embodiment of medical device 100 showing force sensors 102 positioned at distal ends of the middle finger, index finger and thumb of the glove body of the medical device 100. In addition, FIGS. 3A and 3B show at least a part of the circuitry or wiring 110 required for the functioning of the sensors 102 and for the transmission of the sensed data to the wrist enclosure 106 for storage and further processing. As shown in FIG. 3B, the wires 110 extend between the sensors 102 and the wrist enclosure 106 and can run along one side of the glove body, such as the back side of the glove and hand.

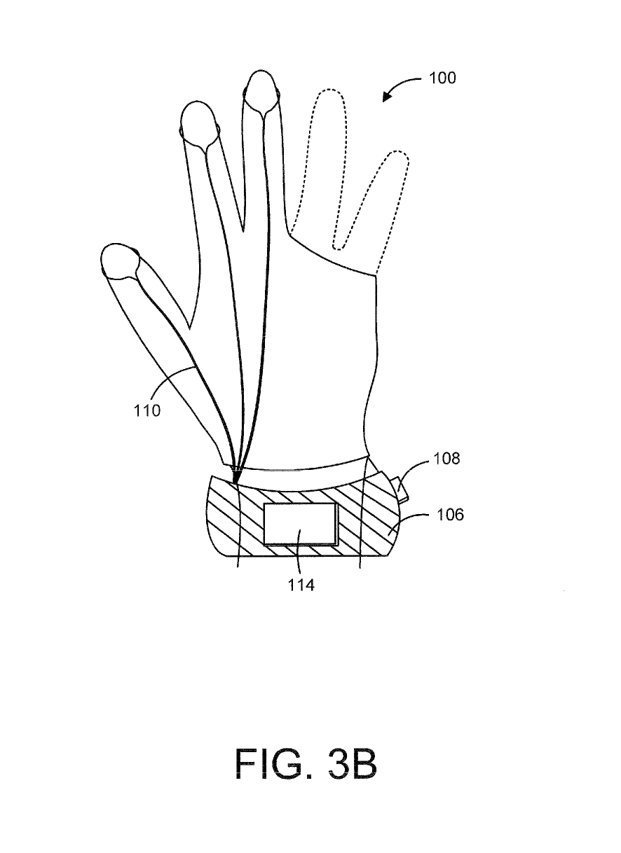

FIG. 4 shows an embodiment of medical device 100 including nanotechnology sensors 112. The nanotechnology sensors 112 can be used to assist in detecting tissue stiffness and cancerous tissue in the patient and can be distally positioned along a finger covering of the glove body.

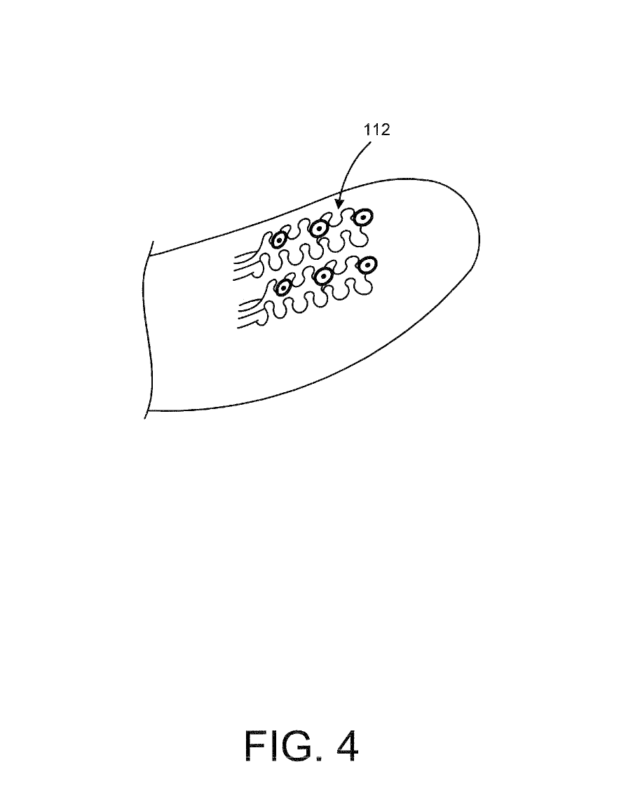

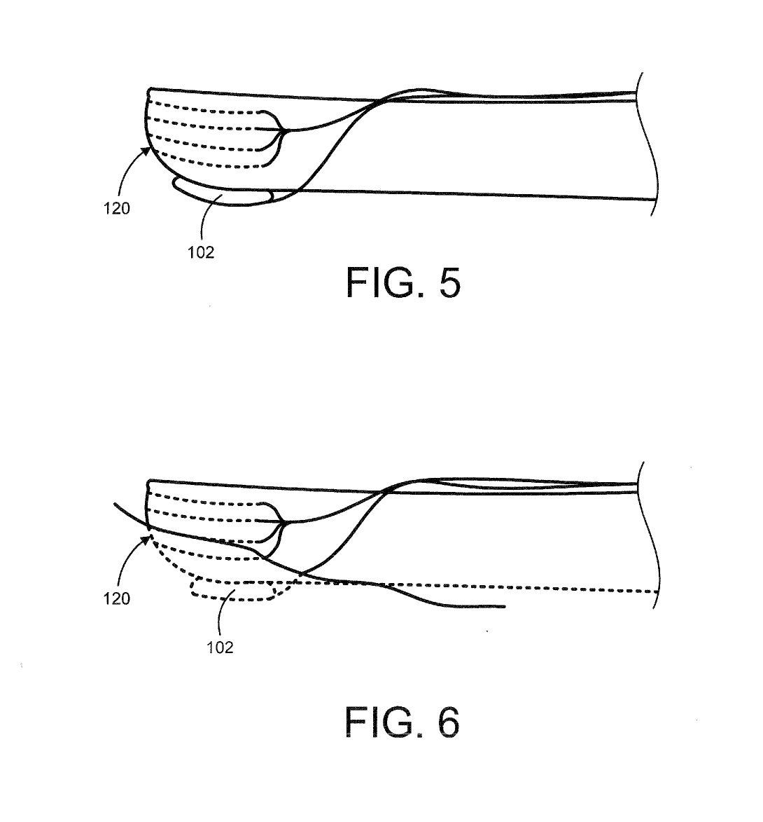

FIG. 5 shows an embodiment of the medical device including circumferential pressure sensors 120 located along a part of the distal end, or fingertip, of the glove body. The circumferential pressure sensors 120 can be comprised of one or more pressure sensors positioned a defined distance apart from each other in a circumferential arrangement around a distal end of the glove, or finger. The circumferential pressure sensors 120 can be arranged such that as the user presses the glove, or finger, into the body of a patient, as shown in FIG. 6, the more pressure sensors come into contact with the patient due to the user's finger becoming impressed into the patient's body. Therefore, as the user's finger becomes increasingly impressed into the body of the patient, the circumferential pressure sensors 120 will be able to detect the amount of displacement the user's finger has made into the body of the patient by evaluating the number of circumferential pressure sensors 120 contacting the body of the patient and factoring in the displacement between the circumferential pressure sensors 120. In addition, the medical device can include one or more processors for computing and evaluating the sensed data, including from the circumferential pressure sensors 120.

In at least some embodiments of the medical device, the data obtained from the force sensors 102 can be computed and evaluated in conjunction with the displacement of the tissue being compressed, or impressed, such as described above using circumferential pressure sensors 120, in order to detect the presence or absence of tissue masses under the skin. In particular, medical device 100 can use such sensed data to detect breast cancer, testicular cancer, etc., and masses such as enlarged lymph nodes.

At least some advantages of the medical device includes the ability to maintain dexterity of the user, such as a physician or nurse, while still collecting data relating to the tissue being examined, such as tissue stiffness. For example, the glove material of the medical device 100 can include thin spandex, which can facilitate dexterity. In addition, the wrist enclosure 106 can include a plastic housing that can be easily mounted to the operator's wrist and that can contain the necessary circuits, electronics, processors, user inputs and device outputs of the medical device 100.

For example, during a breast exam, a user wearing the medical device 100 can depress the skin of a patient with one or more fingertips, which creates a displacement between the starting position of the finger (i.e., the fingertip placed against the skin of the patient prior to depressing the fingertip against the skin of the patient) and the fully depressed finger. With the assistance of the present medical device 100, a user can perform palpation while the medical device senses the displacement of the one or more fingers into the body or skin of the patient and the amount of force applied against the body or skin of the patient. The medical device can then calculate the associated tissue stiffness using the sensed force from the force sensors 102 and displacement measurements from the circumferential pressure sensors 120.

In some embodiments, the force sensor is centered in the middle of the fingertip, with concentric rings of pressure transducers, such a piezoelectric and peizoresistive fabric, progressing up the edge of the fingertip, such as is shown in FIGS. 5 and 6. In addition, the rings are placed at known distances from the bottom part of the force sensor 102 located on the fingertip, which can allow a combination of force sensing while determining the depth of tissue palpation via detecting enveloping tissue along the edges of the fingertip.

Young's modulus, also known as tensile or elastic modulus, is the measure of the stiffness of an elastic material. In this application it can be used to characterize the stiffness of abnormal and potentially cancerous tissue. Because cancerous tissue masses will typically be stiffer than surrounding normal tissue, detecting this change in tissue stiffness can be used to alert the operator of a possible abnormal mass, which can indicate malignancy. In at least some instances, cancerous tissues can be as much as seven times as stiff as normal tissues.

Equation 1, when used in conjunction with the sensed data collected by the medical device 100, can be used to determine Young's modulus. Referring to equation 1, E represents Young's modulus, F is the force exerted on an object under tension, A₀ is the original cross-sectional area through which the force is applied, ΔL, is the amount by which the length of the object changes, L₀ is the original length of the object.

$$E ≡ \frac{tensile stress}{tensile strain} = \frac{σ}{ɛ} = \frac{F / {A}_{0}}{Δ L / {L}_{0}} = \frac{F {L}_{0}}{{A}_{0} Δ L} \qquad Equation 1$$

The calculated Young's modulus differs based on whether the tissue is cancerous or not. The present medical device 100 is able to calculate the Young's modulus by first determining F based on the applied force of the user, such as from sensed data obtained from the force sensors 102 along the distal end of the glove. In addition, the area of the force sensor 102 on the fingertip is variable A₀, the original cross-sectional area through which the force is applied, which may also be equivalent to the area of the fingertip of the glove.

In order to obtain ΔL and L₀, it is necessary to determine how far the fingertip moves (ΔL), as well as the starting length of the material compressed (L₀). In some embodiments, this calculation can be determined with the assistance of an external camera in order to obtain two images of the tissue. For example, the camera 104 shown in FIG. 1 can record at least a pre-palpation image and a post-palpation image, which can be used to determine the displacement of the finger against the tissue.

Alternatively, camera 104 can be mounted on an examining table and can record all operations involving the glove of the medical device, including the motion and displacement associated with palpation. The recorded displacements can then be used to calculate the stiffness, or elastic modulus, of the patient's tissue. In addition, the medical device 100 can include a feedback signal from the camera, which can indicate if the camera's view is being obscured in order to assure correct function of the device, such as capturing the impression of the finger into the examined tissue.

In some embodiments, the medical device 100 can be configured to determine tissue stiffness as well as obtain sensed data from other secondary sensors, including one or more of a thermometer or accelerometer. For example, a thermometer can sense the temperature of the skin being palpated, which may indicate an underlying pathological process or condition.

In addition, an accelerometer mounted on the palmar aspect of the glove of the medical device 100 can allow the medical device 100 to self-locate the glove body relative to the patient body, such as relative to one or more reference points, such as the bellybutton and bilateral axilla. This method requires calibration of the medical device 100 prior to use, and may be able to determine not only the tissue stiffness as previously described, but a rough sense of where the glove body is located relative to the patient's body.

In some embodiments of the medical device 100, an LCD screen can be mounted on the wrist enclosure 106 and can provide some user feedback related to tissue stiffness. The user feedback related to tissue stiffness can be provided on either the LCD screen or on a computer after having connected the wrist enclosure 106 to a computer and transferred the data stored on the wrist enclosure 106.

Although a few specific embodiments have been described in detail above, other modifications consistent with the spirit of this disclosure are contemplated.

1. A medical device comprising:
a glove body configured to fit over a hand of a user and at least one force sensor secured to at least one fingertip of the glove body;
a displacement assessment device configured to collect data indicating displacement of at least one fingertip as it is pressed against tissue in order to determine the stiffness of the tissue; and
a processor configured to process data obtained by the displacement assessment device and the at least one force sensor to assess the stiffness of the tissue in contact with the at least one force sensor.

2. The medical device of claim 1, wherein the displacement assessment device is a camera.

3. The medical device of claim 2, wherein the camera is coupled to the glove body.

4. The medical device of claim 1, wherein the displacement assessment device is a circumferential pressure sensor array.

5. The medical device of claim 4, wherein the circumferential pressure sensor array is located along at least one fingertip of the glove body and is comprised of a plurality of pressure sensors positioned a defined distance apart in a circumferential arrangement relative to the fingertip.

6. The medical device of claim 5, wherein the pressure sensors include piezoelectric and resistive transducers.

7. The medical device of claim 5, wherein the processor determines the displacement of the finger against the tissue by evaluating the number of pressure sensors that are in contact with the tissue and the displacement between each pressure sensor.

8. The medical device of claim 4, wherein the circumferential pressure sensor array is comprised of concentric rings of pressure sensors progressing up the edge of the fingertip and positioned a defined distance apart from each other and which at least partially encircle a centrally located force sensor on the fingertip.

9. The medical device of claim 1, wherein the processor is configured to determine the elastic modulus of the tissue.

10. The medical device of claim 1, further comprising a wrist enclosure configured to contain the processor.

11. The medical device of claim 10, further including a display mounted on the wrist enclosure and configured to display information related to the stiffness of the tissue.

12. The medical device of claim 1, wherein an accelerometer is mounted on the glove body and provides data to the processor to determine the location of the glove body relative to one or more reference points along the body of the patient.

13. The medical device of claim 2, wherein the camera records at least a pre-palpation image and a post-palpation image.

14. A method of using a medical device for obtaining data characterizing stiffness of tissue, comprising:
securing a medical device to a hand of a user, wherein the medical device includes a glove body configured to fit over a hand of a user with at least one force sensor secured to at least one fingertip of the glove body, a displacement assessment device configured to collect data indicating the displacement of at least one fingertip as it is pressed against tissue in order to determine the stiffness of the tissue, and a processor configured to process data obtained by the displacement assessment device to assess the stiffness of the tissue in contact with the at least one force sensor;
placing at least one force sensor against the tissue of a patient;
collecting data associated with the force sensor and data associated with displacement assessment device;
processing the data; and
determining the stiffness of the tissue.

15. The method of claim 14, wherein the displacement assessment device is a camera.

16. The method of claim 15, wherein the camera is coupled to the glove body.

17. The method of claim 14, wherein the displacement assessment device is a circumferential pressure sensor array.

18. The method of claim 17, wherein the circumferential pressure sensor array is located along at least one fingertip of the glove body and is comprised of a plurality of pressure sensors positioned a defined distance apart in a circumferential arrangement relative to the fingertip.

19. The method of claim 18, wherein the pressure sensors include piezoelectric and resistive transducers.

20. The method of claim 18, wherein the processor determines the displacement of the finger against the tissue by evaluating the number of pressure sensors that are in contact with the tissue and the displacement between each pressure sensor.

21. The method of claim 17, wherein the circumferential pressure sensor array is comprised of concentric rings of pressure sensors progressing up the edge of the fingertip and positioned a defined distance apart from each other and which at least partially encircle a centrally located force sensor on the fingertip.

22. The method of claim 14, wherein the processor is configured to determine the elastic modulus of the tissue.

23. The method of claim 14, wherein the medical device further comprises a wrist enclosure configured to contain the processor.

24. The method of claim 23, wherein the medical device further comprises a display mounted on the wrist enclosure and configured to display information related to the stiffness of the tissue.

25. The method of claim 14, wherein an accelerometer is mounted on the glove body and provides data to the processor to determine the location of the glove body relative to one or more reference points along the body of the patient.

26. The method of claim 15, wherein the camera records at least a pre-palpation image and a post-palpation image.
